(19) United States
(12) Patent Application Publication
(10) Pub. No.: US 2001/0038832 A1
(43) Pub. Date: Nov. 8, 2001

(54) Nitric oxide and analogues thereof effectuate sensitization of neoplasm and immunologically undesired tissues to cytotoxicity

(76) Inventors: Benjamin Bonavida, Los Angeles, CA (US); Hermes Garban, Los Angeles, CA (US)

Correspondence Address: MILORD & ASSOCIATES, 2029 CENTURY PARK EAST, SUITE 1700, LOS ANGELES, CA, 90067

(21) Appl. No.: 09/833,539

(22) Filed: Apr. 11, 2001

Related U.S. Application Data
(60) Provisional application No. 60/196,210, filed on Apr. 11, 2000.

(51) Int. Cl. A61K 38/19; A61K 38/21; A61K 33/00

(52) U.S. Cl. 424/85.5; 424/600; 424/85.1; 514/27; 514/283; 514/171; 514/449

(57) ABSTRACT

A method for treatment of conditions in a patient concerns the treatment of cancers, infectious diseases, and unwanted tissues by interferon-gamma (IFN-γ), Nitric Oxide (NO), NO donors, or inducible nitric oxide synthase (iNOS), applied either individually or in combination. In addition, a method for treating a cancer, infectious diseases, and immunologicaly unwanted tissues in an individual by administering a therapeutically effective amount of NO, NO donors, or iNOS thereby inducing the cancer cells to undergo Fas and TNF receptor family-mediated cytotoxicity which may also be combined with the administration of immunotherapeutic and/or cytotoxic agents.

### RELATED APPLICATION

This application claims priority from U.S. provisional patent application No. 60/196,210, filed on Apr. 11, 2000 and entitled “NITRIC OXIDE AND ANALOGUES THEREOF EFFECTUATE SENSITIZATION OF NEOPLASM AND IMMUNOLOGICALLY UNDESIRED TISSUES TO CYTOTOXICITY,” the contents of which are incorporated herein by reference.

### FIELD OF THE INVENTION

The present invention is related to novel methods for disease therapy and for sensitization therapy of resistant cells to apoptosis in general. In particular, the present invention is related to the methods of sensitizing abnormal cells, such as cancer cells, and/or infected cells to apoptosis through the use of the following agents, or analogues either individually or in combination: IFN-γ, nitric oxide, nitric oxide donors, nitric oxide inducers, and nitric oxide analogues.

### BACKGROUND OF THE INVENTION

Apoptosis, also known as programmed cell death, is an important physiological process in multicellular organisms. Apoptosis permits the elimination of cells that are produced in excess, that are no longer necessary, that have developed improperly, that have sustained genetic damage, or that have been negatively altered. Apoptosis occurs in a plurality of different tissue systems and must be properly regulated to provide optimal benefits to the organism. Alternatively, dysregulation of the apoptotic mechanism can result in the development of significant disease which results from either inhibition of and/or inappropriate cell death. Therefore, it is extremely important and useful to determine the mechanisms by which apoptosis is achieved.

For example, apoptosis is mediated, at least in part, by a cell surface receptor protein, Fas, which plays an important role in the development and function of the immune system and is well documented in the prior art. Apoptosis can be induced by the binding and cross-linking of Fas with either anti-Fas agonist antibody or with Fas-ligand (FasL). The Fas receptor (CD95/APO-1) has been recognized as a central receptor for apoptosis, particularly in mediating nonspecific T-cell cytotoxicity and activation-induced cell death (AICD) in the peripheral immune system. However, Fas/Apo-1 (CD95³) expression is not restricted to cells of the hematopoietic lineage. It has been observed that the Fas receptor is expressed constitutively in the normal folliculi of the ovary, endometrial gland cells of the uterus and columnar epithelium of the uterine cervix.

It has been shown that ovarian surface epithelial cells (OSE) are especially susceptible to Fas-mediated apoptosis among the normal mouse corpora lutea (CL) cultures containing luteal, stromal, endometrial cells and fibroblasts and also OSE exposed to the Fas-agonist antibody JO2. In vivo, OSE undergo programmed cell death before ovulation and rapidly proliferate to repair the surface of the ovulatory follicle after ovulation. It is noteworthy that most ovarian cancers are derived from the OSE.

It is known that the majority of ovarian carcinoma cell lines screened for Fas expression were positive and their sensitivity to the anti-Fas agonist antibody did not correlate with the level of Fas expression. Furthermore, the down-regulation of Fas expression and subsequent resistance to anti-Fas were observed in many tumor types such as, but not limited to, the drug-resistant human ovarian-carcinoma IGR-OVI/VCR, the human breast-carcinoma cell line MCF7Adr and the leukemic lymphoblast CEM/VLB cells, suggesting that the alteration of Fas expression following drug-resistance is not restricted to one cell type.

It is known that IFN-γ treatment sensitizes several Fas-resistant tumor cell lines, including the ovarian carcinoma cell line AD10 to Fas-mediated cytotoxicity and apoptosis. IFN-γ treatment of carcinoma cell lines results in upregulation of Fas antigen expression with IFN-γ-mediated sensitization to Fas killing. It is also known that IFN-γ alone or in combination with TNF-α, Interleukin 1β (IL-1β) and bacterial lipopolysaccharide (LPS) can induce the expression of nitric oxide synthase type II (iNOS) in a wide variety of tissue organs and in some tumor cell lines. The inducible type of nitric oxide synthase (iNOS) is considered to be a central molecule in the regulation of the immune response to tumors. Further, iNOS is responsible for the production of large amounts of nitric oxide (NO).

Nitric oxide is a potent and pleiotropic free radical molecule that has been involved in a wide variety of physiological and pathophysiological functions. NO is generated in low levels by two constitutive nitric oxide synthases (eNOS and nNOS) and in much greater levels by the inducible NO synthase (iNOS). It has been a long debate about the specific role that nitric oxide might play in apoptosis. The prior art has heretofore shown that nitric oxide is an inhibitor of apoptosis in direct contrast to the disclosure of the present invention.

For instance, endogenous NO synthesis or exposure to low level of NO donors was first shown to inhibit apoptosis in human B lymphocytes, and similar findings have been reported in splenocytes, eosinophils, and endothelial cells. NO donors have also been directed toward the specific disruption of the Fas-induced apoptotic mechanism. Basal NOS activity in human leukocytes was shown to inhibit Fas-induced apoptosis via a cGMP-independent mechanism and further inhibition of caspase activation.

Therefore, there remains a need for determining the role of NO, either endogenous or exogenous, in sensitizing certain tumor cells to Fas mediated apoptosis and for therapeutic treatments of certain tumor cells therewith.

### SUMMARY OF THE INVENTION

Accordingly, the primary objective of the present invention is to overcome the limitations of the prior art.

Another object of the invention is to provide methods for treating individuals having drug and/or immuno resistant diseases such as cancer and infectious diseases.

It is another object of the present invention to provide a method for treating individuals having cancer or infectious diseases, wherein the method offers a mechanism for achieving better therapeutic efficacy than the current standard therapies for cancer.

It is another object of the present invention to provide a method for treating individuals having cancer or infectious diseases, wherein the method offers a mechanism for achieving better therapeutic efficacy than the current standard therapy for cancer, and further offers a mechanism to minimize toxicity to the treated individual.

It is a further object of the present invention to provide a method for treating individuals having cancer or infectious diseases, wherein the method offers a mechanism for achieving better therapeutic efficacy than the current standard therapy for cancer, and further offers a mechanism to minimize toxicity to the treated individual, wherein the method is facilitated by Fas-mediated cytotoxicity of the malignant tumor.

The foregoing objects are based on a novel discovery that cancer cells which are exposed to NO, iNOS, NO donors or mimics thereof, express significant amounts of Fas on their cell-surface, and further, that ligation (cross-linking) of the receptor on these Fas-bearing tumor cells leads to rapid programmed cell death of the treated tumor. In another embodiment of the present invention, the method comprises combination therapy involving the use of one or more compositions for ligation of Fas expressed by the cancer cells in conjunction with one or more compositions which potentiates or enhances Fas-mediated cytotoxicity of the treated tumor, such as NO, iNOS, NO donors or mimics thereof, such that they upregulate the cell-surface expression of Fas by the tumor.

Such stated objects and advantages of the invention are only examples and should not be construed as limiting the present invention. These and other objects, features, aspects, and advantages of the invention herein will become more apparent from the following detailed description of the embodiments of the invention when taken in conjunction with the accompanying figures and the claims that follow.

### BRIEF DESCRIPTION OF THE FIGURES

It is to be understood that the drawings are to be used for the purposes of illustration only and not as a definition of the limits of the invention.

FIG. 1A. illustrates the iNOS expression in AD10 ovarian carcinoma cells after treatment thereof by IFN-γ.

FIG. 1B. illustrates that nitrites and nitrates increase in the culture medium after AD10 ovarian carcinoma cells are treated with IFN-γ.

FIG. 2A. illustrates the Fas-mediated cytotoxicity of A2780 cells treated with IFN-γ and Fas-agonistic antibody CH11 in increasing concentrations.

FIG. 2B. illustrates the Fas-mediated cytotoxicity of AD10 cells treated with IFN-γ and Fas-agonistic antibody CH11 in increasing concentrations.

FIG. 3A. illustrates that the sensitization of the A2780 cell line to Fas-mediated cytotoxicity was not affected by the addition of a NOS inhibitor.

FIG. 3B. illustrates that the sensitization of the AD10 cell line to Fas-mediated cytotoxicity was affected by the addition of a NOS inhibitor.

FIG. 4A. illustrates that the A2780 cell line was sensitized to Fas-mediated cytotoxicity with the addition of a NO donor.

FIG. 4B. illustrates that the AD10 cell line was sensitized to Fas-mediated cytotoxicity with the addition of a NO donor.

FIG. 5. illustrates the ability of NO to sensitize AD10 cells, treated with IFN-γ, to Fas-mediated apoptosis wherein (a) represents the control group; (b) represents cells treated with CH11 alone; (c) represents cells treated with NO and CH11; (d) represents cells treated with NOS inhibitor; (e) represents cells treated with the NO donor SNAP and CH11; and (f) represents cells treated with NO only.

FIG. 6A. illustrates the upregulation of Fas receptor expression on AD10 cells by increasing concentrations of SNAP.

FIG. 6B. illustrates that Fas receptor mRNA expression was reduced by incubation of the cells with the NOS inhibitor.

FIG. 6C. illustrates an upregulation in Fas receptor protein level for AD10 cells treated with IFN-γ except in the presence of NOS inhibitor, but was restored in the presence of SNAP.

FIG. 6D. illustrates that IFN-γ treated AD10 and A2780 cells had increased expression of Fas surface molecule, which was reduced in the presence of NOS inhibitor, but was restored by incubation with the NO donor.

### DETAILED DESCRIPTION OF THE PREFERRED EMBODIMENTS

A. Definitions

“Gamma interferon”, “interferon-gamma”, or “IFN-γ” refers variously to all forms of (human and non-human animal) gamma interferon that are shown to be biologically active in any assay, whether obtained from natural sources, chemically synthesized or produced by techniques of recombinant DNA technology. As IFN-γ is known to be highly species specific, in animal experiments, IFN-γ of the animal species to be treated is preferably employed.

“Therapeutically effective amount” of IFN-γ, in a pharmacological sense, in the context of the present invention refers to an amount effective in the treatment of cancer, infectious diseases, and other diseases that are responsive to the administration thereof.

“Treatment” refers to both therapeutic treatment and prophylactic or preventative measures, wherein the object is to prevent or slow down the disease in general, and cancer in particular. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial antihypertrophic effect for an extended period of time.

“Administered in combination with” one or more further theapeutic agents includes simultaneous (concurrent) and consecutive administration in any order.

“Administered in known methods” includes, for purposes of illustration but not limitation, injection or infusion by intravenous, intraperitoneal, intracerebral, intramuscular, intraocular, intraarterial, or intralesional routes, or by sustained-release systems, or orally, or topically, or an aerosol formulation suitable for intranasal or intrapulmonary delivery.

B. One Preferred Embodiment for Carrying Out the Invention

Nitric Oxide (NO), analogues, and mimics thereof sensitize resistant tumor cells to endogneous or exogenous agents such as, but not limited to, biological, chemical, pharmaceutical, radiological, and immune mediated cytotoxic agents. An example of neoplasm are ovarian carcinoma cells and human prostate carcinoma cell lines. These Fas-resistant tumor cells may be treated with NO, NO analogues, and mimics thereof, either individually or in combination, to achieve Fas-induced apoptosis. Synergy is achieved with the Fas-ligand, Fas agonist antibodies and/or cytokines, either endogenous or exogenous, and other compositions which enhance Fas-mediated cytotoxicity. For purposes of illustration, but not limitation, such compositions may be human or recombinant cytokines, either endogenous or exogenous, such as, but not limited to, intleukin-1β (IL-1β), interferon-α (IFN-α), interleukin-2 (IL-2), interferon-γ (IFN-γ), and tumor necrosis factor-α (TNF-α), or compositions or combinations of compositions such as adriamycin, cisplatin, diptheria toxin, or any other therapeutic drugs that are known in the art. In addition, the treatment of carcinoma cells with NO, NO analogues, and/or mimics results in sensitization thereof to the TNF receptor superfamily.

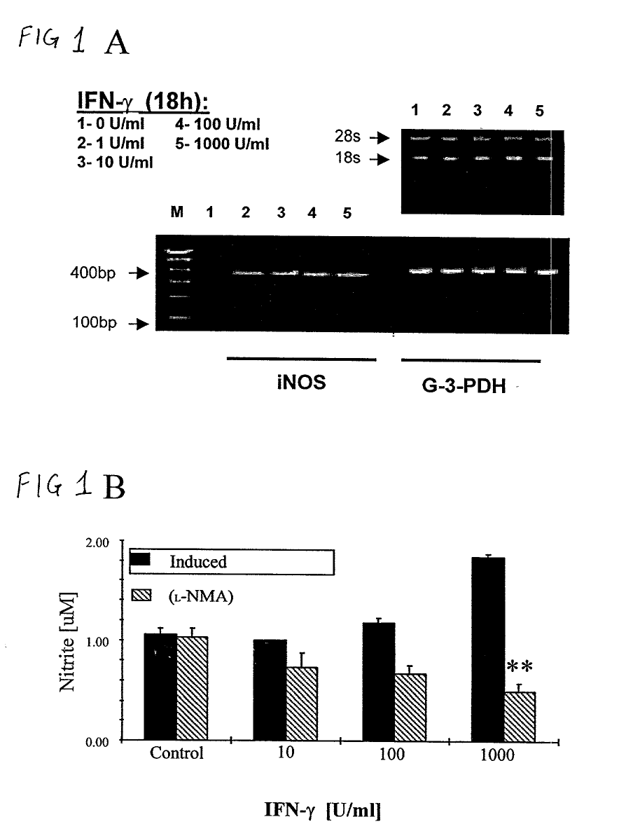

Now referring to FIGS. 1A and B, ovarian carcinoma cell line AD10 can be induced to generate iNOS upon treatment with IFN-γ. AD10 cells (5×10⁵/well) were cultured for 24 hours in 6-well plates supplemented with 1% FCS prior to incubation with increasing concentrations of human recombinant IFN-γ (0 U/ml to 1000 U/ml) for 18 hours. Significant levels of iNOS mRNA was observed by semiquantitative RT-PCR in 1 U/mL of IFN-γ-treated AD10 cells (FIG. 1A). iNOS mRNA levels increased as a function of increasing concentrations of IFN-γ. A plateau of iNOS mRNA expression was reached with 100 U/mL of IFN-γ. In contrast to AD10, it was not possible to detect any iNOS expression upon IFN-γ treatment of the parental A2780 ovarian carcinoma cell line (data not shown). These results demonstrate the inducibility of functional iNOS in the AD10 cell line.

The activity of iNOS and generated NO were monitored by the release of NO₂⁻/NO₃⁻ into the cell culture medium as determined by the Griess reaction. Production of NO was measured as accumulation of nitrites and nitrates in the culture medium of AD10 cells (2×10⁵/well in 12-well plates) under the same conditions as in FIG. 1A in the presence or absence of the NOS inhibitor L-NMA (1.0 mM). This activity was demonstrated to be specific for NOS by blocking the generation of NO₂⁻/NO₃⁻ using the NOS inhibitor L-NMA (1.0 mM) prior to the induction in AD10 by IFN-γ (FIG. 1B). Thus, the ovarian carcinoma cell line AD10 can be induced by IFN-γ to produce NO by iNOS, whereas the ovarian carcinoma cell line A2780 is not able to express iNOS and cannot generate NO upon IFN-γ stimulation.

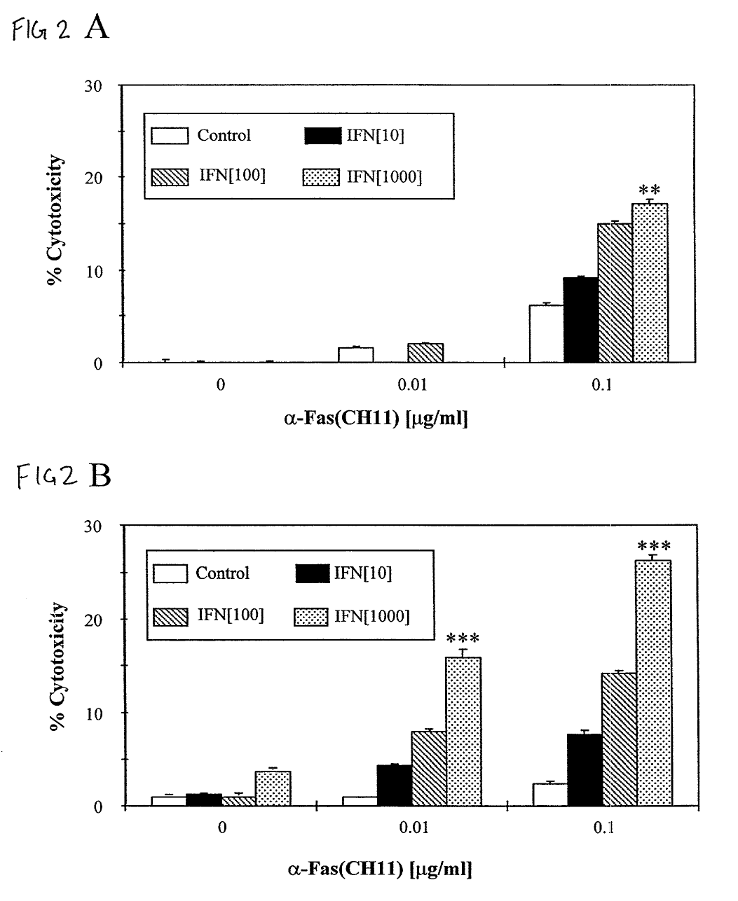

Now referring to FIGS. 2A and B, illustrated therein is the sensitivity of the ovarian carcinoma cell line A2780 and the adriamycin resistant subline AD10 to Fas-mediated apoptosis using the Fas agonistic antibody CH11 in a preferred embodiment. However, it is to be understood that alternate Fas agonistic antibodies, such as, but not limited to, Mabs M2 and M3 (IgG), anti-Fas Mab (IgM), anti-APO-1 (IgG), cytotoxic lymphocytes and macrophages, recombinant Fas-ligand may also be used.

In a preferred embodiment, A2780 and AD10 cells (1×10⁴ cells/well) were cultured in 96-well plates supplemented with 10%FCS and pretreated with increasing concentrations of IFN-γ (0, 10, 100 and 1000 U/mL) 18 h prior to performing the cytotoxicity assay with increasing concentrations of the anti-Fas agonist antibody CH11 (0, 0.01, and 0.1 μg/mL). The parental cell line A2780, as illustrated in FIG. 2A, exhibited a lower capacity of being sensitized by IFN-γ when compared with the adriamycin resistant AD10 cell line, as illustrated in FIG. 2B. IFN-γ-pretreated AD10 cells, as seen in FIG. 2B, were sensitized an average of 10 fold higher compared with the untreated control group.

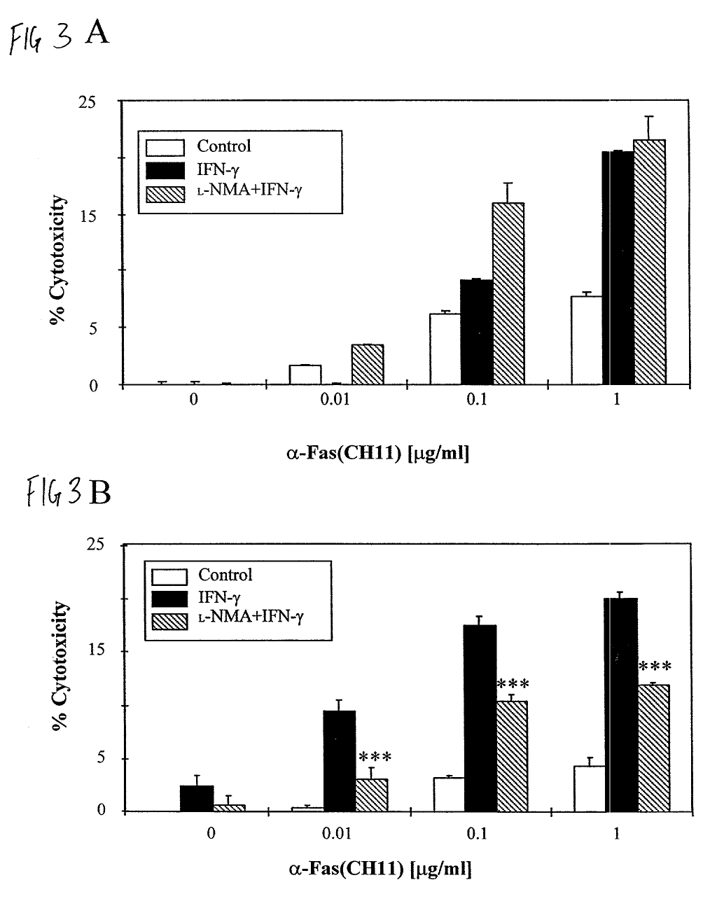

IFN-γ alone or in combination with other proinflammatory cytokines such as, but not limited to, TNF-α, IL-1 and LPS, have been shown to be effective in the induction of the inducible form of nitric oxide synthase (iNOS) in several tumor cell lines. Now also referring to FIGS. 3A and B, therein the role of iNOS in the mechanism of sensitization to Fas-mediated cytotoxicity of ovarian carcinoma cells is illustrated. Ovarian carcinoma cells, A2780 and AD10, were respectively incubated in the presence or in the absence of the competitive NOS inhibitor L-NMA (1 mM) 6 h prior to IFN-γ treatment. NOS inhibition significantly decreased IFN-γ-mediated sensitization to Fas-mediated apoptosis in AD10 cells, as illustrated in FIG. 3B, suggesting that iNOS induction by IFN-γ is an important component in the process of sensitization. In contrast, A2780 cells did not respond to NOS blocking and preserved their sensitization to Fas-mediated apoptosis achieved with IFN-γ treatment, as illustrated in FIG. 3A. Accordingly, the inducibility of the ovarian carcinoma cell line AD10 to generate NO by iNOS and the susceptibility of this cell line to be sensitized to the Fas-mediated apoptosis is hereby demonstrated. Furthermore, it is herein illustrated that the parental cell line A2780, which is not able to express iNOS upon IFN-γ stimulation, was not sensitized at the same level when compared with the adriamycin resistant cell line AD10, which is induced by IFN-γ alone to express iNOS.

The role of the endogenously generated NO in the IFN-γ-mediated sensitization to Fas-induced apoptosis was corroborated by the use of an exogenous source of NO, which mimics the production of NO by iNOS. Ovarian carcinoma cells were cultured in the presence or in the absence of three different NO donors; namely sodium nitroprusside (SNP), S-Nitroso-N-acetylpenicillamine (SNAP) and DETA NONOeate (NOC18), in equimolar concentrations of 10 and 100 μM for 24 h before the cytotoxicity assay with the Fas-agonistic antibody. It is to be understood that although specific NO donors are listed above, those are by way of example and not limitation, and therefore, alternate agents that can exert the same chemical, cellular, or genetic alteration and function as NO may be used to sensitize certain carcinomas to Fas mediated apoptosis.

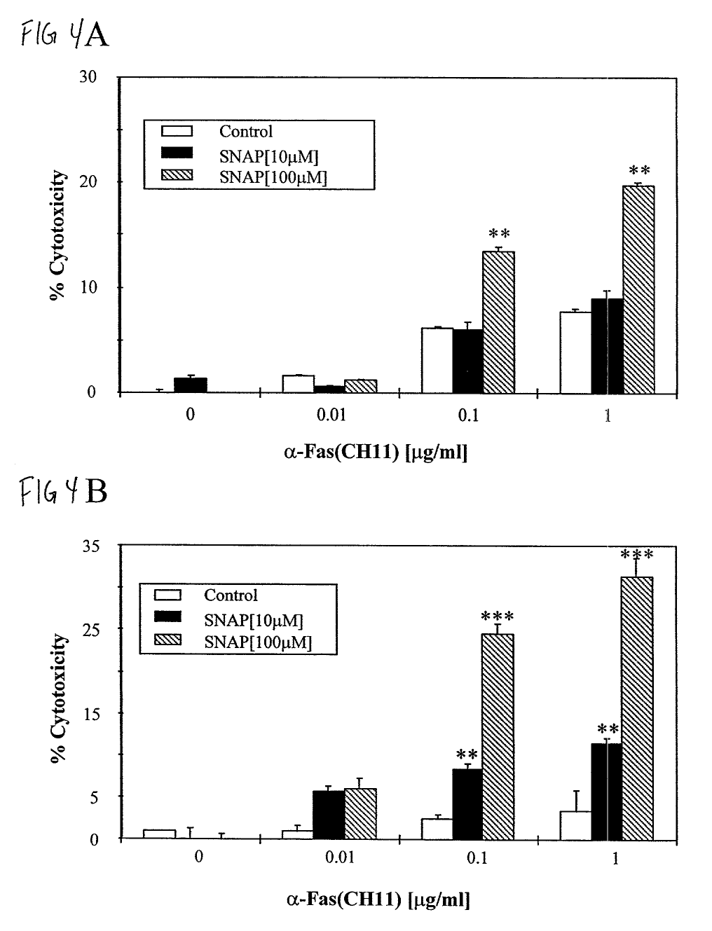

In all cases, NO donors sensitized ovarian cells to Fas-mediated cytotoxicity in the same way as IFN-γ-mediated sensitization. The findings with SNAP are shown in FIGS. 4A and B. Cultured A2780, as illustrated in FIG. 4A, and AD10 cells, as illustrated in FIG. 4B, (1×10⁴ cells/well) in 96-well plates supplemented with 10% FCS were incubated in the presence of the photoactivated SNAP (0, 10, and 100 μM) for 24 hours and cytotoxicity of the Fas-agonist antibody (CH11) was assessed by LDH release into the culture medium. These results establish a correlation between the generation of NO, either endogenously by IFN-γ and iNOS or exogenously, and sensitization of ovarian carcinoma cell lines to Fas-mediated cytotoxicity.

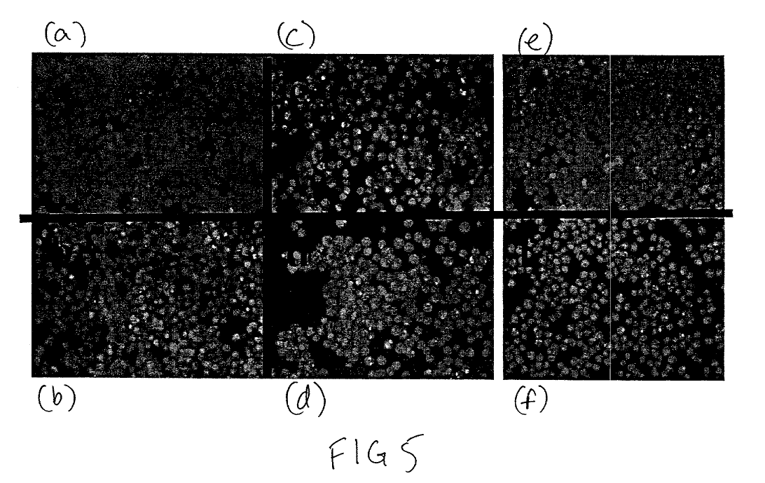

Now referring to FIGS. 5a through f, sensitization of AD10 to CH11 (Fas-ligand agonist)-mediated cytotoxicity was due to apoptosis. The morphological pattern was examined by staining the ovarian carcinoma cells with acridine orange/ethidium bromide (AO/EB). Pretreated cells with IFN-γ for 18 h and then treatment with 0.1 μg/mL of the Fas-agonistic antibody CH11 for 6 h, resulted in a greater frequency of cells undergoing apoptosis (FIG. 5C) as compared with the untreated control group (FIG. 5B). When the cells were incubated in the presence of the NOS inhibitor L-NMA (1 mM) 6 h prior to IFN-γ pretreatment, the frequency of cells undergoing apoptosis (FIG. 5D) was significantly reduced and was less than that of cells incubated in the absence of the NOS inhibitor. Furthermore, treatment of AD10 cells with SNAP prior to exposure to CH11 also increased the frequency of cells undergoing characteristic apoptosis (FIG. 5F), whereas the addition of the NO donor alone did not increase the number of apoptotic cells (FIG. 5E). It is to be understood that other NO donors or analogues thereof may also be used to sensitize the AD10 cells to Fas mediated apoptosis by a plurality of Fas agonists.

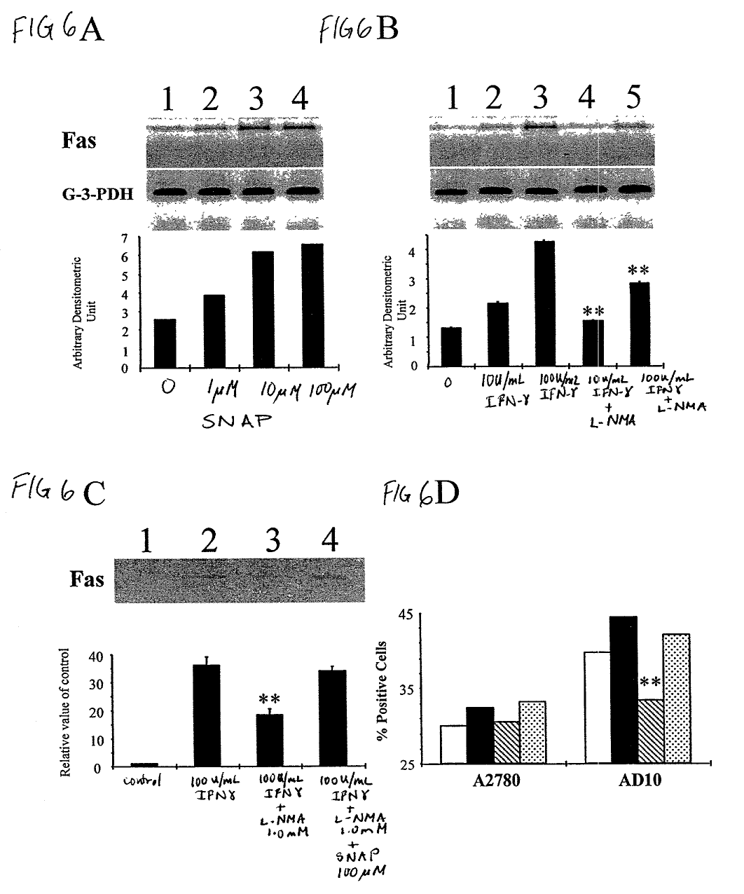

Now referring to FIG. 6A through D, the role of NO in the regulation of Fas receptor expression in AD10 was determined. First, AD10 cells (5×10⁵/well cultured in a 12-well plate supplemented with 10% FCS) were exposed to increasing concentrations of a NO donor (SNAP: 0, 1, 10 and 100 μM) for 18 h and the relative Fas mRNA expression was determined by RT-PCR. Clearly, SNAP upregulated Fas receptor mRNA expression as illustrated in FIG. 6A. It is to be understood that other NO donors and analogues thereof may be substituted for SNAP while producing augmentation of Fas receptor expression with increasing concentrations.

Now referring to FIG. 6B, endogenous generation of NO by the induction of iNOS is responsible for the upregulation of Fas. AD10 cells were treated with 10 and 100 U/mL of IFN-γ in the presence or absence of the NOS inhibitor L-NMA (1.0 mM) for 24 hours. Fas mRNA expression was markedly reduced by NOS blocking (FIG. 6B).

Now referring to FIG. 6C, the upregulation of Fas receptor was corroborated by Western blot. IFN-γ induced AD10 cells (1×10⁷/plate) were cultured in a 100-mm plate supplemented with 10% FCS. IFN-γ mediated upregulation of Fas protein level was markedly reduced in the presence of the NOS inhibitor and restored by the addition of SNAP. It is to be understood that other NO donors and analogues thereof may be substituted for SNAP while producing augmentation of Fas receptor expression with increasing concentrations.

Now referring to FIG. 6D, increased expression of Fas surface molecule was detected by flow cytometry on AD10 (2×10⁵ cells/well) cultured in 12-well plates supplemented with 10% FCS in the presence of IFN-γ (10 U/mL) for 18 hours (solid bars) when compared with untreated control cells (blank bars). This increased expression was partially blocked by the NOS inhibitor (slashed bars) and restored by incubation with NO donors in general, and in a preferred embodiment, where the NO donor is SNAP. Altogether, these results demonstrate a strong correlation between the generation of NO by ovarian and prostate carcinoma cells and upregulation of Fas receptor expression.

Accordingly, it has been herein illustrated that the AD10 ovarian carcinoma cell line, when stimulated with IFN-γ, can express iNOS and produce NO. The generation of NO correlates with the sensitization of AD10 cells to Fas-induced apoptosis and can be blocked by the NOS inhibitor, thus implicating the role of NO in the IFN-γ-mediated sensitization to Fas-induced killing. Moreover, the use of NO donor bypassed the inability of the parental cell line A2780 to express iNOS and sensitized those cells to the Fas agonist antibody. Sensitization was concomitantly observed with upregulation of Fas gene expression. In contrast to the role of NO in protecting against apoptosis in cells of the hematopoietic lineage, our findings demonstrate that NO plays a role in the sensitization of tumor cells to Fas-mediated apoptosis. Such sensitization is due to the regulation of Fas gene expression and/or signaling towards apoptosis.

Furthermore, human prostate carcinoma cell lines may also be treated with NO, NO analogues, mimics, and/or derivatives thereof to achieve apoptosis in a similar fashion as described above. In particular, the PC-3 prostate carcinoma cell line produced results that were similar to the AD10 cell line, and the DU-145 prostate carcinoma cell line produced results that were similar to the A2780 cell line (results not shown).

Thus, NO generation (NO-based therapies) can be used to control tumor cell death by apoptotic-mediated mechanisms. A drawback to systemic therapies is the lack of selectivity in delivering therapy to the intended target, diseased tissue, rather than to normal tissue. Accordingly, a pharmaceutically acceptable carrier known in the prior art may be used to deliver a composition which enhances Fas-mediated cytotoxicity to a uniquely targeted cell. In the present case, NO, NO analogues, mimics, and/or derivatives thereof may be pharmaceutically delivered to a specific carcinoma cell type to increase sensitization thereof in general, and specifically, to upregulate Fas expression. Furthermore, NO, in combination with compositions which enhance Fas-mediated apoptosis, can render selective and targeted therapy while eliminating or at least decreasing unintended damage to surrounding cells.

The following examples further illustrate the present invention and, of course, should not be construed as in any way limiting its scope, but rather providing at least one preferred embodiment for practicing the same.

### EXAMPLE 1

This example describes the cell cultures and lines used in one embodiment of the present invention. The AD10 cell line is an adriamycin resistant, MDR phenotype-expressing, subline derived from the ovarian carcinoma cell line A2780 and both were obtained from Dr. Ozols (Fox Chase Cancer Center, Philadelphia, Pa.). Cell cultures were maintained as monolayers on plastic dishes in DMEM or medium (Life Technologies, Bethesda, Md.), supplemented with 10% heat-inactivated FCS (Life Technologies, Bethesda, Md.), 1% L-glutamine (Life Technologies, Bethesda, Md.), 1% pyruvate (Life Technologies, Bethesda, Md.), 1% nonessential amino acids (Life Technologies, Bethesda, Md.) and 1% fungi-bact solution (Irvine Scientific, City, State). The cells were preincubated with the iNOS inhibitor, N^{G}-Monomethyl-L-arginine (L-NMA; final concentration 1 mM; Sigma Chemical Co., St. Louis, Mo.) or an equimolar concentration of its biologically inactive D-enantiomer, D-NMA (Sigma Chemical Co., St. Louis, Mo.) for 18 h prior to IFN-γ induction.

### EXAMPLE 2

This example describes one preferred method of conducting a Reverse Transriptase Polymerase Chain Reaction (RT-PCR) in order to determine cell gene expression. Total RNA was extracted and purified from approximately 5×10⁵ cells for each different condition by a single step guanidinium thiocyanate-chloroform method with STAT 60™ reagent (Tel-Test “B”, Inc., Friendswood, Tex.). 1 μg of total RNA was reverse transcribed to first stranded cDNA for 1 h at 42° C. with SuperScript™ II reverse transcriptase [200 U] and random hexamer primers [20 μM] (Life Technologies, Bethesda, Md.). Amplification of 1/10 of these cDNA by PCR was performed using the following gene-specific primers: Fas receptor sense (5′-ATG CTG GGC ATC TGG ACC CT-3′), Fas receptor antisense (5′-GCC ATG TCC TTC ATC ACA CAA-3′) [338 bp expected product]. iNOS sense (5′-CCG AGC CCG AAC ACA CAG AAC-3′) and iNOS antisense (5′-GGG TTG GGG GTG TGG TGA TGT-3′) [462 bp, expected product]. Internal control for equal cDNA loading in each reaction was assessed using the Following genespecific glyceraldehyde-3-phosphate dehydrogenase (G-3-PDH) primers: G-3-PDH sense (5′-GAA CAT CAT CCC TGC CTC TAC TG-3′), G-3-PDH antisense (5′-GTT GCT GTA GCC AAA TTC GTT G-3′) [355 bp expected product]. PCR amplifications were carried out using the Hot Start/Ampliwax method as described by the supplier (Perkin Elmer) with the following temperature cycling parameters: 94° C./45 sec; 65° C./2 min for 26 cycles and a final extension at 72° C./10 min. The amplified products were resolved by 1.5% agarose gel electrophoresis and their relative concentrations were assessed by densitometric analysis (BIOSOFT, Cambridge, UK.) of the ethidium bromide (EtBr)-stained image.

### EXAMPLE 3

This example describes one preferred method of separating and sorting cells through fluorescence-activated cell sorting (FACS). Surface Fas antigen expression on tumor cells was determined by flow cytometry. Briefly, harvested cells were washed with cold buffer consisting of PBS without Ca⁺⁺ or Mg⁺⁺ with 2% heat-inactivated FCS and 0.1% sodium azide. 2×10⁵ cells per sample were pretreated with human AB serum (Gemini Bioproducts, Calabasas, Calif.) for 1 h, washed twice and resuspended in 50 μl of PBS. The cells were incubated with 10 μg/mL of anti-Fas monoclonal antibody FITC-conjugated (PharMingen, San Diego, Calif.) or isotype control antibody for 1 h. The cells were then washed twice and fixed in 2% paraformaldehyde solution (Sigma Chemical Co., St. Louis, Mo.) and flow cytometry was conducted all the FACScan facility of the UCLA Department of Microbiology and Immunology.

### EXAMPLE 4

This example describes a preferred method of determining cytotoxicity. Sensitization to Fas-mediated apoptosis was assessed using the agonist anti-Fas monoclonal antibody CH11(IgM) [0.01, 0.1 and 1 μg/mL] (Kamiya Biomedical, Thousand Oaks, Calif.) in a 24 h incubation assay. The lactate dehydrogenase (LDH)-based CytoTox 96™ Assay (Promega, Madison, Wis.) was used to determine cytotoxicity. Briefly, 1×10⁴ cells/sample, in quadruplicate, were distributed into a 96-well flat-bottom microtiter plate (Costar, Cambridge, Mass.). After the initial incubation for each different experimental condition, released LDH into the culture supernatants was measured with a 30-minute coupled enzymatic assay which results in the conversion of a tetrazolium salt (INT) into a red formazan product that is read at 490 nm in an automated plate reader (Emax, Molecular Devices, Sunnyvale, Calif.). Percentage cytotoxicity was calculated using the spontaneous release-corrected OD as follows: % cytotoxicity=(OD of experimental well/OD of maximum release control well)×100.

### EXAMPLE 5

This example describes one preferred method of determining nitrate and nitrite concentrations. Nitric oxide generation was monitored indirectly by levels of nitrite/nitrate (NO₂⁻/NO₃⁻) released into the culture medium as determined by the diazotization reaction of Griess with NaNO₂ as standard Briefly, 50 μl aliquots of cell culture supernatants from each sample were mixed with one volume of Griess reagent [1% sulfanilamide; 0.1% naphthylethylene diamine dihydrochloride; 2.5% H₃PO₄] and incubated at room temperature for 10 min. The absorbance at 550 nm was measured in an automated plate reader (Emax, Molecular Devices, Sunnyvale, Calif.). Nitrite concentrations were calculated by comparison with OD₅₅₀ values of standard solutions of sodium nitrite prepared in culture medium.

### EXAMPLE 6

This example describes one preferred method of determining protein expression. Cell extracts for iNOS and Fas receptor analysis were prepared by lysing 5×10⁶ cells in 1 mL phosphate buffer solution [10 mM EDTA, 1% triton X-100, 1 mM Phenylmethylsulfonyl fluoride (PMSF) and 0.01% leupeptin]. Cell lysates were boiled (3 min) with 1 volume gel loading buffer [50 mM Tris/10% sodium dodecyl sulfate (SDS)/10% glycerol/10% 2-mecaptoethanol/2 mg/mL bromophenol blue] and centrifuged at 1×10⁴ g for 10 min. Protein concentrations of the supernatants were determined according to Bradford, and total protein equivalents for each sample were separated on 12% SDS-polyacrylamide minigels (Bio-Rad, Richmond, Calif.) and transferred to nitrocellulose membranes (Amersham Corp., Arlington Heights, Ill.). Nonspecific immunoglobulin G (IgG) binding sites were blocked with 5% dried milk protein, and samples were then incubated with the antibody to iNOS [1:1000] (Transduction Laboratories, Lexington, Ky.) or CD95 [1:500] (PharMingen, San Diego, Calif.). Relative concentrations were assessed by densitometric analysis (BIOSOFT, Cambridge, UK) of the bands detected using a horseradish peroxidase-conjugated secondary antibody coupled to ECLchemiluminescent system (Amersham Corp.).

### EXAMPLE 7

This example describes one preferred embodiment for determining apoptosis through acridine orange/ethidium bromide staining. Characteristic apoptotic morphological changes were assessed by fluorescent microscopy using the acridine-orange and ethidium bromide staining (AO/EB) method. Briefly, adherent cells, under different experimental conditions, were cultured in 24 wells plates, and washed with PBS once prior to staining. Monolayers of adherent cells were covered with 100 μl of AO/EB solution (4 μg/mL of each). Immediately after adding the AO/EB solution, each sample was examined under an inverted/fluorescent microscope.

While the above description contains many specificities, these should not be construed as limitations on the scope of the invention, but rather as an exemplification OF one preferred embodiment thereof. Many other variations are possible without departing from the essential spirit of this invention. Accordingly, the scope of the invention should be determined not by the embodiment illustrated, but by the claims and their legal equivalents in the non-provisional application.

What is claimed is:

1. A method for the treatment of at least neoplastic diseases and infectious diseases in a multicellular organism, comprising:
administering an effective amount IFN-γ; and
an effective amount of a sensitizing agent.

2. The method of treatment of claim 1, wherein said sensitizing agent is selected from a group consisting of at least NO, iNOS, NO donors, and NO mimics.

3. The method of treatment of claim 2, wherein said neoplastic disease is a cancer.

4. The method of treatment of claim 3, wherein said cancer is ovarian cancer.

5. The method of treatment of claim 3, wherein said cancer is prostate cancer.

6. The method of treatment of claim 3, wherein said cancer is resistant to a treatment selected from a group consisting of at least chemotherapy, immunotherapy, and radiation therapy.

7. The method of treatment of claim 3, wherein a chemotherapeutic agent is administered therewith.

8. The method of treatment of claim 7, wherein said chemotherapeutic agent is selected from the group consisting of at least Amethopterin, Ara-C, BCNU, Bleomycin, Cladribine, Cyclophophamide, Dactinomycin, Doxorubicin (Adriamycin), DTIC, Etoposide, 5-FU, Floxuridine, Hydrea, Idamycin, Ifosfamide, Levamisole, Mechlorethamine hydrochloride, Medroxyprogesterone, Megestrol Acetate, Melphalan, Mesna, Mitomycin-C, Octreotide acetate, Paraplatin, Prednisone, Retinoic acid, Streptozocin, Tamoxifen, Taxol, Thio-TEPA, Vinblastine, and Vincristine.

9. The method of treatment of claim 3, wherein said cancer is characterized by resistance to Fas ligand and further TNF family members.

10. The method of treatment of claim 3, wherein a member is included therewith and increases Fas mediated cytotoxicity.

11. The method of treatment of claim 10, wherein said member is selected from the group consisting of an agonist anti-Fas monoclonal antibody, Fas-ligand, and combination thereof.

12. The method of treatment of claim 10, wherein said member is a cytokine seleted from the group consisting of IL-2, IL-10, IL-1β, TNF-α.

13. A method of combination therapy for treating cancer cells in a multicellular organism by inducing the cancer cells to undergo Fas-mediated cytotoxicity, the method comprising:
administering at least one composition which enhances Fas-mediated cytotoxicity; and
administering at least one Fas-cross-linking member selected from the group consisting of an agonist anti-Fas monoclonal antibody, fragments thereof, soluble Fas-ligand, and a combination thereof;
administering at least one sensitizing agent.

14. The method of combination therapy of claim 13, wherein said sensitizing agent is selected from a group consisting of at least NO, iNOS, NO donors, and NO mimics.

15. The method of combination therapy of claim 13, wherein the composition which enhances Fas-mediated cytotoxicity is a cytokine selected from the group consisting of IFN-γ, IL-2, IL-10, IL-1β, TNF-α.

16. The method of combination therapy of claim 13, further comprising the step of administering a chemotherapeutic agent selected from the group consisting of Amethopterin, Ara-C, BCNU, Bleomycin, Cladribine, Cyclophophamide, Dactinomycin, Doxorubicin (Adriamycin), DTIC, Etoposide, 5-FU, Floxuridine, Hydrea, Idamycin, Ifosfamide, Levamisole, Mechlorethamine hydrochloride, Medroxyprogesterone, Megestrol Acetate, Melphalan, Mesna, Mitomycin-C, Octreotide acetate, Paraplatin, Prednisone, Retinoic acid, Streptozocin, Tamoxifen, Taxol, Thio-TEPA, Vinblastine, and Vincristine.

17. The method of combination therapy of claim 13, wherein said cancer has become resistant to chemotherapy and immunotherapy.

18. The method of combination therapy of claim 14, wherein the composition which enhances Fas-mediated cytotoxicity is a cytokine selected from the group consisting of IFN-γ, IL-2, IL-10, IL-1β, TNF-α.

19. The method of combination therapy of claim 14, further comprising the step of administering a chemotherapeutic agent selected from the group consisting of Amethopterin, Ara-C, BCNU, Bleomycin, Cladribine, Cyclophophamide, Dactinomycin, Doxorubicin (Adriamycin), DTIC, Etoposide, 5-FU, Floxuridine, Hydrea, Idamycin, Ifosfamide, Levamisole, Mechlorethamine hydrochloride, Medroxyprogesterone, Megestrol Acetate, Melphalan, Mesna, Mitomycin-C, Octreotide acetate, Paraplatin, Prednisone, Retinoic acid, Streptozocin, Tamoxifen, Taxol, Thio-TEPA, Vinblastine, and Vincristine.

20. A method of treating cancerous cells that are resistant to conventional therapy, the method comprising:
inducing the cancerous cell to undergo Fas-mediated cytotoxicity;
administering systemically a sensitizing agent selected from the group consisting of NO, iNOS, NO donors, and NO mimics;
administering systemically a composition which enhances Fas-mediated cytotoxicity and is selected from the group consisting of IFN-γ, IL-2, IL-10, IL-1β, TNF-α; and
administering systemically a Fas-cross-linking member selected from the group consisting of an agonist anti-Fas monoclonal antibody, fragments thereof, soluble Fas-ligand.
